(19) United States
(12) Patent Application Publication
(10) Pub. No.: US 2026/0285785 A1
(43) Pub. Date: Sep. 24, 2026

(54) BINARY POLYETHYLENEIMINE EMULSION BREAKER COMPOSITIONS FOR ETHYLENE PLANT QUENCH WATER SYSTEMS

(71) Applicant: Ecolab USA Inc., Saint Paul, MN (US)

(72) Inventors: Muhammad Tariq Khan, Thuwal (SA); Kameswara Vyakaranam, Sugar Land, TX (US); Satheesh Kumar Sambandam, Richmond, TX (US); Ashish Dhawan, Aurora, IL (US); Deepak Sharma Shivaprasad, Dubai (AE)

(21) Appl. No.: 19/563,569

(22) Filed: Mar. 11, 2026

Related U.S. Application Data
(60) Provisional application No. 63/773,650, filed on Mar. 18, 2025.

(51) Int. Cl. C07C 7/11; B01D 17/04; C08G 73/02

(52) U.S. Cl. CPC C07C 7/11; B01D 17/047; C08G 73/024

(57) ABSTRACT

A binary polyethylenimine composition can be used to effectively treat quench water for an ethylene production process across a wide range of pH conditions observed in typical production to inhibit and/or break an emulsion in the quench water. The binary polyethylenimine composition includes a first hyperbranched polyethylenimine having a first molecular weight and a second hyperbranched polyethylenimine having a second molecular weight greater than the first molecular weight.

### RELATED APPLICATION

This application claim priority to U.S. Provisional Application No. 63/773,650, filed Mar. 18, 2025, the entire contents of which are incorporated herein by reference.

### TECHNICAL FIELD

This disclosure relates to compositions and techniques for inhibiting and/or breaking emulsions in hydrocarbon/water systems, particularly quench water generated in an ethylene production process.

### BACKGROUND

Ethylene, a fundamental building block in the petrochemical industry, is primarily produced through the thermal cracking of hydrocarbons, such as naphtha or ethane. This high-temperature process generates a complex mixture of products, including ethylene, propylene, and other valuable olefins, as well as undesirable byproducts like methane, hydrogen, and heavier hydrocarbons. Following the cracking reaction, the hot effluent stream must be rapidly cooled to stop the ongoing reactions and recover the desired products. This cooling process is commonly accomplished using a quench system.

The quench system typically employs a large volume of water, often referred to as quench water, to directly contact and cool the cracked gas. This direct contact serves not only to reduce the temperature of the gas stream but also to condense heavy hydrocarbons and other high-boiling components. The quench water system plays a critical role in the overall efficiency and stability of the ethylene production process.

However, the quench process introduces several challenges. The complex mixture of hydrocarbons and other compounds present in the cracked gas can lead to the formation of stable emulsions within the quench water. These emulsions are dispersions of one liquid (typically hydrocarbons) within another (water), stabilized by the presence of surface-active agents. These surface-active agents can be inherent in the cracked gas, formed as byproducts during the cracking process, or even intentionally added to the quench water system.

The formation of emulsions in the quench water can have detrimental effects on the ethylene production process. For example, emulsions can reduced heat transfer efficiency by increasing the viscosity of the quench water and inhibiting efficient heat removal from the cracked gas, potentially leading to operational instability and reduced product yields. Emulsion formation can also cause fouling and plugging problems, depositing on heat exchanger surfaces, piping, and other equipment. The presence of hydrocarbons and other contaminants in the emulsified quench water complicates wastewater reclamation and disposal. In severe cases, carryover of emulsified hydrocarbons into downstream processing units can contaminate the desired ethylene product stream. For these and other reasons, management and control of emulsion formation in ethylene production quench water systems is a crucial aspect of efficient and reliable plant operation.

### SUMMARY

In general, this disclosure is directed to compositions and techniques for treating liquid streams containing mixtures of hydrocarbons and water to inhibit and/or break the emulsions formed between the oleaginous and aqueous components of the stream. While the disclosed compositions may be used in a variety of hydrocarbon/water streams, the compositions can be particularly beneficial for treating a quench water stream used during a hydrocarbon production process, particularly an ethylene production process. Ethylene production involves thermally cracking (e.g., steam cracking) of hydrocarbons like naphtha or ethane, to produce target olefins like ethylene and propylene, alongside byproducts such as methane, hydrogen, and heavier hydrocarbons. The hot effluent stream generated through thermal cracking is cooled by a quench system to halt further reactions and enable product recovery, typically by direct contact with a quench water. The quench water, laden with these condensed hydrocarbons and other byproducts, may be continuously circulated and treated with compositions according to the disclosure to inhibit and/or break emulsions that may otherwise form in the quench water.

The disclosed compositions for treating emulsions in hydrocarbon/water streams are formed of two different hyperbranched polyethylenimines, thereby providing a binary polyethylenimine composition. The binary polyethylenimine composition include a first hyperbranched polyethylenimine having a first molecular weight and a second hyperbranched polyethylenimine having a second molecular weight greater than the first molecular weight. In some implementations, the second molecular weight is at least one order of magnitude greater than the first molecular weight (10×), such as at least two orders of magnitude greater (100×). In either case, the binary polyethylenimine composition includes a first hyperbranched polyethylenimine having a comparatively low molecular weight and a second hyperbranched polyethylenimine having a comparatively high molecular weight.

Applicant has identified that the disclosed binary polyethylenimine composition can be effective a low dosage and perform over a wide pH range, delivering performance flexibility and emulsion control over a range of operational pH conditions that may be experienced in different production environments. The disclosed binary polyethylenimine composition can exhibit better performance than the individual polyethylenimine components forming the binary polyethylenimine composition, providing an unexpected performance synergy between the polyethylenimine components over the individual components along. This performance synergy is exhibited across a range of pH conditions and dosage concentrations for the binary polyethylenimine composition.

In some implementations, each of the individual polyethylenimine components forming the binary polyethylenimine composition is a non-alkoxylated hyperbranched polyethylenimine. For example, each of the individual polyethylenimine components forming the binary polyethylenimine composition may be characterized by having high primary and tertiary amine fractions, attributable to branching, and a lower secondary amine fraction (e.g., compared to the combined primary and tertiary amine fractions), which shows linearity.

In one example, a method of treating ethylene plant quench water is described. The method involves adding a binary polyethylenimine composition to a quench water for an ethylene production process thereby inhibiting and/or breaking an emulsion in the quench water. The method specifies that the binary polyethylenimine composition includes a first hyperbranched polyethylenimine having a first molecular weight and a second hyperbranched polyethylenimine having a second molecular weight greater than the first molecular weight.

In another example, an emulsion treatment composition for an ethylene plant quench water is described. The emulsion treatment composition includes a first hyperbranched polyethylenimine having a first molecular weight and a second hyperbranched polyethylenimine having a second molecular weight greater than the first molecular weight.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a block diagram illustrating an example ethylene production process utilizing a quench water that may be treated with binary polyethylenimine compositions according to the disclosure.

FIGS. 2 and 3 are bar charts showing the turbidity removal performance of an example binary polyethylenimine composition compared to alternative emulsion treatment chemistry options across a range of dosage concentrations.

### DETAILED DESCRIPTION

This disclosure relates to binary polyethylenimine compositions that include a comparatively high molecular weight hyperbranched polyethylenimine and a comparatively low molecular weight hyperbranched polyethylenimine along with techniques for using such binary polyethylenimine composition to treat emulsions in combined hydrocarbon and water streams. While the disclosed binary polyethylenimine compositions can be used in any desired emulsion treatment applications, the compositions may be beneficially used for treating quench waters, such as those used in ethylene production processes. Additional details on binary polyethylenimine compositions according to the disclosure will be described in greater detail below. However, details on example processes that can generate a quench water desirably treated using compositions according to the disclosure will first be described.

Thermal cracking of hydrocarbons is a petrochemical process is widely used to produce olefins such as ethylene, propylene, butylene, butadiene, and aromatics such as benzene, toluene, and xylenes. Each of these is a valuable commercial product in its own right. For instance, the olefins may be oligomerized (e.g., to form lubricant basestocks), polymerized (e.g., to form polyethylene, polypropylene, and other plastics), and/or functionalized (e.g., to form acids, alcohols, aldehydes and the like), all of which have well-known intermediate and/or end uses.

Steam cracking, also referred to as pyrolysis, has long been used to crack various hydrocarbon feedstocks into olefins, preferably light olefins such as ethylene, propylene, and butene. Typically, the feedstock for steam cracking is a hydrocarbon such as naphtha, gas oil, or other non-resid containing fractions of whole crude oil, which may be obtained, for instance, by distilling or otherwise fractionating whole crude oil. Conventional steam cracking utilizes a steam cracking furnace which has two main sections: a convection section and a radiant section. The hydrocarbon feedstock typically enters the convection section of the furnace as a liquid (except for light feedstocks which enter as a vapor) wherein it is typically heated and vaporized by indirect contact with hot flue gas from the radiant section and by direct contact with steam. The vaporized feedstock (and optional steam) mixture is then conveyed into the radiant section where the cracking takes place. Typically the vaporized mixture is introduced through crossover piping into the radiant section where it is quickly heated, at pressures typically ranging from about 10 to about 50 psig (69 to 345 kPa), to a severe hydrocarbon cracking temperature, such as in the range of from about 1450° F. (788° C.) to about 1650° F. (900° C.), to provide thorough thermal cracking of the feed stream. The resulting products, including olefins, leave the steam cracking furnace for further downstream processing.

For example, following cracking, the effluent from the reactor can be rapidly cooled to a temperature at which no additional reaction occurs. This rapid cooling may be effected by indirectly cooling the effluent in a transfer line exchanger(s) which generates high pressure steam and then further directly cooled by circulating water created from condensation of steam within a quench water tower. The effluent from the reactor contains a variety gaseous hydrocarbons, e.g., saturated, monounsaturated, and polyunsaturated, and can be aliphatic and/or aromatic, as well as significant amounts of molecular hydrogen.

For gaseous feedstocks (e.g., ethane, propane and butanes), a quench oil tower (QOT) may not be required because only small amounts of liquids containing five or more carbons liquids may be produced. For these feedstock types, a simple quench water tower may be used to cool the effluent gas from the transfer line exchanger. In either case, the cracked gas may be cooled in the by quench oil tower by direct contact with quench water. Typically, the bottoms stream leaving the quench tower feeds an oil-water separator drum, which function as a three-phase separator, with a light hydrocarbon phase that floats on water, and the tar which sinks in water, as the bottom phase, and water as the middle phase. The quench water leaving the oil-water separator drum may contain residual fine solid particles, unsettled free oil, emulsified oil, and dissolved hydrocarbons. The majority of this quench water may be recirculated for low-level heat recovery within the plant before returning to the quench water tower. Binary polyethylenimine compositions according to the disclosure may be added to the quench water at any desired one or more locations along the process flow to inhibit the formation of an emulsion between the hydrocarbon and water phases and/or to partially or full break an emulsion formed between the hydrocarbon and water phases.

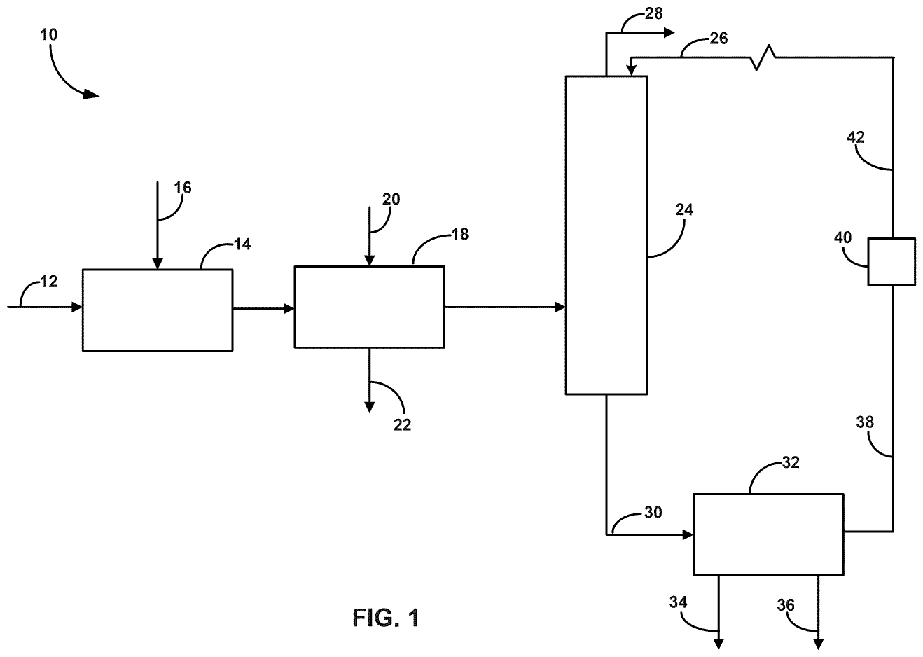

FIG. 1 is a block diagram illustrating an example ethylene production process 10 utilizing a quench water that may be treated with binary polyethylenimine compositions according to the disclosure. In the example of process 10, gaseous hydrocarbon feedstock 12 (e.g., ethane, propane, and/or and other trace hydrocarbons) can be delivered to a convection section in the upper part of a cracking furnace 14. Gaseous hydrocarbon feedstock 12 may be first preheated in hydrocarbon preheat banks, not illustrated, and/or mixed with steam 16. The hydrocarbon and steam mixture may then be further preheated sequentially in a hydrocarbon dilution steam bank before leaving the convection section to cross over to the radiant coil inlets.

The heat required for the cracking reaction may be supplied by a radiant section in the lower part of the cracking furnace 14. In some applications, the radiant section of the furnace may include floor fired burners. In either case, the burner firing rate can be controlled to achieve an average coil outlet temperature. The resulting effluent from furnace 14 may be rapidly cooled in quench exchangers 18. Quench exchangers 18 may include any suitable heat exchangers such as, for example, double pipe exchangers and/or horizontal shell and tube transfer line exchanger (TLE) quench exchangers. Boiler feed water 20 may be supplied to quench exchanger 18 to generate steam 22 for use in other processes.

The heat exchanger effluent including cracked gasses may then be fed to a quench water tower 24. The heat exchanger effluent can be cooled by direct countercurrent contact with quench water 26 in the quench water tower. The cooled gas from the quench water tower overheads 28 may then be transported to a cracked gas compressor. A water and hydrocarbon mixture 30 can be collected in the bottom of the quench water tower, which may then be transported to an oil-water separator 32.

Oil-water separator 32, which can also be referred to as a quench water settler, can separate a heavy phase, comprising tar and coke particles, from the bulk stream. A water-hydrocarbon interface may be established that allows for separation of the remaining two phases. In the case of cracking an ethane feed, light tars may be produced which may have a specific gravity close to that of water. The tar yield may be high enough to cause the water leaving oil-water separator 32 to cause downstream fouling of the quench circuit. Accumulated tar and coke particles 34 may settle out from the quench water in oil-water separator 32, which may be stored or sent to an incinerator for disposal. A pyrolysis gasoline stream 36 may also be removed from oil-water separator 32. Pyrolysis gasoline is a naphtha boiling range product with a high aromatics content that may be produced as a side product of the thermal cracking reaction. The hot raw quench water 38 from the oil-water separator 32 may be circulated through heat exchanger(s) 40 (e.g., to recover heat and/or cooling water exchanges to cool the water) to produce a recovered quench water 42. Recovered quench water 42 can be returned to quench water tower 24 as part of a quench water loop (in which quench water is used, recovered, and recycled).

Binary polyethylenimine compositions according to the disclosure can be added to quench water to inhibit the formation of an emulsion and/or to break a formed emulsion, such as at quench water 26, oil-water separator 32, hot raw quench water 38, and/or recovered quench water 42 in the example of FIG. 1. The binary polyethylenimine compositions can be used as emulsion breakers in other desired applications as well. In either case, the binary polyethylenimine compositions includes two different hyperbranched polyethylenimines having different molecular weights: a comparatively high molecular weight hyperbranched polyethylenimine and a comparatively low molecular weight hyperbranched polyethylenimine. Each of the hyperbranched polyethylenimines can be non-halogenated (halogen free) and can be non-ethoxylated.

Polyethylenimine (PEI) or polyaziridine is a cationic polymer with repeating units composed of an amine group and two carbon aliphatic CH₂CH₂ spacers between each amine group. Linear polyethyleneimines contain all secondary amines. By contrast, branched polyethylenimine which contain primary, secondary and tertiary amino groups. The degree of branching can be controlled by the reactions condition employed (e.g., temperature, concentration, duration) and further functionalization can be achieved through chemical post-modification (e.g., amine alkylation, acylation, condensation, sulfonylation, etc.). Binary polyethylenimine compositions according to the disclosure include two different hyperbranched polyethylenimines.

The extent of branching of the first and second hyperbranched polyethylenimines forming the binary polyethylenimine composition can be dictated, at least in part, by the number of primary and tertiary amines as a percentage of the total number of amines in each polyethylenimine consistent component of the binary composition. Primary and tertiary amines contribute to branching whereas secondary amines contribute to linearity in each polyethylenimine composition. Hyperbranched refers to the polyethylenimine having a significant number of primary and tertiary amines.

The first and second hyperbranched polyethylenimines forming the binary polyethylenimine composition can each include primary amines, secondary amines, and tertiary amines. A primary amine has one carbon-containing group (alkyl or aryl group) attached to the nitrogen atom; a secondary amine has two carbon-containing groups attached to the nitrogen atom; a tertiary amine has three carbon-containing groups attached to the nitrogen atom. The distribution of different amines in each of the first and second hyperbranched polyethylenimines forming the binary polyethylenimine composition can be characterized by the distribution percentage of primary, secondary, and tertiary amines in each of the first and second hyperbranched polyethylenimines which, when added together, constitute all of the amines in each hyperbranched polyethylenimine.

Each hyperbranched polyethyleneimine can include a primary amine fraction that is the molar percentage of primary amines in the hyperbranched polyethylenimine as a percentage of the total number of amines in the hyperbranched polyethylenimine multiplied by 100 (molar number of primary amine groups/(total molar number of primary, secondary, and tertiary amine groups))×100. Each hyperbranched polyethylenimine can exhibit a primary amine fraction that is at least 15%, such as at least 20%, at least 25%, at least 30%, at least 31%, at least 32%, or at least 35%. While each hyperbranched polyethylenimine can exhibit a primary amine fraction that has any one of the foregoing minimum values, the primary amine fraction may simultaneously be less than 50%, such as less than 45%, less than 40%, less than 38%, less than 35%, less than 33%, or less than 30%. For example, the primary amine fraction may range from 20% to 50%, such as from 25% to 45%, from 30% to 40%, or from 32% to 37%.

The first hyperbranched polyethylenimine having a comparatively low molecular weight and the second hyperbranched polyethylenimine having a comparatively high molecular weight may have the same or different primary amine fractions. In some implementations, the first hyperbranched polyethylenimine having a comparatively low molecular weight may have a higher primary amine fraction than the second hyperbranched polyethylenimine having a comparatively high molecular weight. For example, the primary amine fraction of the first hyperbranched polyethylenimine may be at least 1% greater than the primary amine fraction of the second hyperbranched polyethylenimine, such as at least 2% greater, at least 3% greater, at least 4% greater, or at least 5% greater. With any one of these foregoing lower limits, the primary amine fraction of the first hyperbranched polyethylenimine may be less than 15% greater than the primary amine fraction of the second hyperbranched polyethylenimine, such as less than 12% greater, less than 10% greater, less than 9% greater, less than 8% greater, less than 7% greater, or less than 6% greater.

In some applications, the first hyperbranched polyethylenimine having a comparatively low molecular weight can have a primary amine fraction within a range from 35% to 40%, the second hyperbranched polyethylenimine having a comparatively high molecular weight can have a primary amine fraction within a range from 30% to 35%, and the difference in the primary amine fraction between the first hyperbranched polyethylenimine and the second hyperbranched polyethylenimine is within a range from 1% to 10%, such as from 3% to 8%, or approximately 5%.

Each hyperbranched polyethylenimine can also have a secondary amine fraction that is the molar percentage of secondary amines in the hyperbranched polyethylenimine as a percentage of the total number of amines in the hyperbranched polyethylenimine multiplied by 100 (molar number of secondary amine groups/(total molar number of primary, secondary, and tertiary amine groups))×100. Each hyperbranched polyethylenimine can exhibit a secondary amine fraction that is at least 15%, such as at least 20%, at least 25%, at least 30%, at least 31%, at least 32%, at least 35%, or at least 40%. While each hyperbranched polyethylenimine can exhibit a secondary amine fraction that has any one of the foregoing minimum values, the secondary amine fraction may simultaneously be less than 60%, such as less than 50%, less than 45%, less than 43%, less than 42%, less than 41%, less than 40%, less than 39%, less than 38%, less than 37%, less than 36%, or less than 35%. For example, the secondary amine fraction may range from 20% to 50%, such as from 25% to 45%, or 35% to 50%, from 30% to 45%, from 35% to 45%, or from 37% to 41%.

The first hyperbranched polyethylenimine having a comparatively low molecular weight and the second hyperbranched polyethylenimine having a comparatively high molecular weight may have the same or different primary secondary fractions. In some implementations, the first hyperbranched polyethylenimine having a comparatively low molecular weight may have a lower secondary amine fraction than the second hyperbranched polyethylenimine having a comparatively high molecular weight. For example, the primary amine fraction of the second hyperbranched polyethylenimine may be at least 1% greater than the secondary amine fraction of the first hyperbranched polyethylenimine, such as at least 2% greater, at least 3% greater, at least 4% greater, or at least 5% greater. With any one of these foregoing lower limits, the secondary amine fraction of the second hyperbranched polyethylenimine may be less than 15% greater than the secondary amine fraction of the first hyperbranched polyethylenimine, such as less than 12% greater, less than 10% greater, less than 9% greater, less than 8% greater, less than 7% greater, less than 6% greater, or less than 5% greater.

In some applications, the first hyperbranched polyethylenimine having a comparatively low molecular weight can have a secondary amine fraction within a range from 35% to 40%, the second hyperbranched polyethylenimine having a comparatively high molecular weight can have a secondary amine fraction within a range from 40% to 50%, and the difference in the secondary amine fraction between the first hyperbranched polyethylenimine and the second hyperbranched polyethylenimine is within a range from 1% to 10%, such as from 3% to 8%, or approximately 4%.

Each hyperbranched polyethylenimine can also have a tertiary amine fraction that is the molar percentage of tertiary amines in the hyperbranched polyethylenimine as a percentage of the total number of amines in the hyperbranched polyethylenimine multiplied by 100 (molar number of tertiary amine groups/(total molar number of primary, secondary, and tertiary amine groups))×100. Each hyperbranched polyethylenimine can exhibit a tertiary amine fraction that is at least 10%, such as at least 15%, at least 20%, at least 21%, at least 22%, at least 24%, at least 25%, at least 26%, at least 27%, or at least 30% While each hyperbranched polyethylenimine can exhibit a tertiary amine fraction that has any one of the foregoing minimum values, the tertiary amine fraction may simultaneously be less than 40%, such as less than 35%, less than 30%, less than 29%, less than 28%, less than 27%, less than 26%, or less than 25%. For example, the tertiary amine fraction may range from 20% to 50%, such as 20% to 30%, from 24% to 32%, from 25% to 28%, or from 26% to 27%.

The first hyperbranched polyethylenimine having a comparatively low molecular weight and the second hyperbranched polyethylenimine having a comparatively high molecular weight may have the same or different tertiary amine fractions. In some implementations, the first hyperbranched polyethylenimine having a comparatively low molecular weight may have a lower tertiary amine fraction than the second hyperbranched polyethylenimine having a comparatively high molecular weight. For example, the primary amine fraction of the first hyperbranched polyethylenimine may be at least 0.5% greater than the tertiary amine fraction of the second hyperbranched polyethylenimine, such as at least 1% greater, or at least 2% greater. With any one of these foregoing lower limits, the tertiary amine fraction of the second hyperbranched polyethylenimine may be less than 10% greater than the tertiary amine fraction of the first hyperbranched polyethylenimine, such as less than 5% greater, less than 4% greater, less than 3% greater, less than 2% greater, or less than 1% greater.

In some applications, the first hyperbranched polyethylenimine having a comparatively low molecular weight can have a tertiary amine fraction within a range from 23% to 28%, the second hyperbranched polyethylenimine having a comparatively high molecular weight can have a tertiary amine fraction within a range from 25% to 30%, and the difference in the tertiary amine fraction between the first hyperbranched polyethylenimine and the second hyperbranched polyethylenimine is within a range from 0.5% to 5%, such as from 0.5% to 2%, or approximately 1%.

The binary polyethylenimine composition comprises (or consists essentially of, or consists of) a first hyperbranched polyethylenimine having a first molecular weight and a second hyperbranched polyethylenimine having a second molecular weight, where the second molecular weight is greater than the first molecular weight. The first hyperbranched polyethylenimine and the second hyperbranched polyethylenimine may each have a molecular weight within a range from 100 g/mol to 1,000,000 g/mol. The first molecular weight may vary from the second molecular weight by at least one order of magnitude (10×), preferably by at least two orders of magnitude (100×). For example, the second molecular weight may be from 100 to 600 times larger than the first molecular weight, such as from 200 to 500 times larger, or 300 to 400 times larger.

For example, the first molecular weight of the first hyperbranched polyethylenimine may be less than 100,000 g/mol, such as less than 80,000 g/mol, less than 70,000 g/mol, less than 60,000 g/mol, less than 50,000 g/mol, less than 40,000 g/mol, less than 30,000 g/mol, less than 20,000 g/mol, less than 10,000 g/mol, less than 5000 g/mol, less than 4000 g/mol, less than 3000 g/mol, less than 2000, or less than 1500 g/mol. The first molecular weight of the first hyperbranched polyethylenimine may be within a range from 100 g/mol to 50,000 g/mol, such as from 250 g/mol to 25,000 g/mol, from 500 g/mol to 15,000 g/mol, or from 1,000 g/mol to 10,000 g/mol. In some applications, the first hyperbranched polyethylenimine exhibits a first molecular weight within a range from 500 g/mol to 5,000 g/mol, such as from 750 g/mol to 2,500 g/mol, or from 1,000 g/mol to 1,500 g/mol.

The second molecular weight of the second hyperbranched polyethylenimine may be greater than 100,000 g/mol, such as greater than 200,000 g/mol, greater than 300,000 g/mol, greater than 400,000 g/mol, greater than 450,000 g/mol, greater than 500,000 g/mol, greater than 600,000 g/mol, greater than 700,000 g/mol, or greater than 800,000 g/mol. The second molecular weight of the second hyperbranched polyethylenimine may be less than 1.5 million g/mol, such as less than 1.25 million g/mol, less than 1 million g/mol, less than 900,000 g/mol, less than 800,000 g/mol, less than 700,000 g/mol, less than 600,000 g/mol, or less than 500,000 g/mol. In some applications, the second hyperbranched polyethylenimine exhibits a second molecular weight within a range from 100,000 g/mol to 1 million g/mol, such as from 200,000 g/mol to 800,000 g/mol, from 300,000 g/mol to 600,000 g/mol, or from 400,000 g/mol to 500,000 g/mol.

The first and second hyperbranched polyethylenimines forming the binary polyethylenimine composition may each have a general formula H₂N((CH₂)ₓNR₁NH)_{y}NH₂, where x can be 1 to 10, or any number inclusive therein (e.g., 2); and y can be 2 to 1000, or any number inclusive therein). R₁ can be hydrogen (H) or an alkyl group, which may be substituted or unsubstituted. The alkyl group can include a saturated, monovalent unbranched or branched hydrocarbon chain, which may have 1 to 20 carbon atoms. The alky group can include an alkylamine, which can refer to a straight or branched chain alkylamine having 1 to 50 carbon atoms, such as 1 to 20 carbon atoms, for example, CH₂NH₂, CH₂CH₂NH₂, CH₂CH₂CH₂NH₂, CH₂CH(NH₂)CH₃, CH₂CH₂CH₂CH₂NH₂, CH₂CH₂CH(NH₂)CH₃, CH₂CH(NH₂)CH₂CH₃, CH₂CH₂CH₂CH₂CH₂NH₂, CH₂CH₂CH₂CH(NH₂)CH₃, CH₂CH₂CH₂CH(NH₂)CH₃, CH₂CH₂CH(NH₂)CH₂CH₃, CH₂CH(NH₂)CH₂CH₂CH₂CH₃, CH₂CH₂CH₂CH₂CH₂CH₂NH₂, CH₂CH₂CH₂CH₂CH(NH₂)CH₃, CH₂CH₂CH₂CH₂CH(NH₂)CH₃, CH₂CH₂CH₂CH(NH₂)CH₂CH₃, CH₂CH₂CH(NH₂)CH₂CH₂CH₂CH₃, CH₂CH(NH₂)CH₂CH₂CH₂CH₂CH₃, etc. The alkylamine can further include mono- or di-substituted alkyl and/or substituted alkyl chains mentioned above attached to the nitrogen atom of the amine.

The alkyl groups may be devoid of or lack certain heteroatoms, such as halogens (e.g., F, Cl, Br, and/or I), boron, oxygen, sulfur, and/or silicon. Accordingly the first and second hyperbranched polyethylenimines may each be unmodified. Unmodified refers to the polyethylenimine being in basic form and has not undergone any chemical modifications, including alkoxylation, and therefore retains its original structure and properties. An unmodified polyethylenimine may be characterized by a high density of amine groups, making it highly cationic and reactive. Accordingly, the first and second hyperbranched polyethylenimines may each be non-alkoxylated (e.g., meaning that the polyethylenimine is not modified with oxygenated alkyl groups typically prepared by alkoxylation of polyethylenimine with oxygenates).

The binary polyethylenimine composition according to the disclosure can exhibit excellent emulsion inhibiting and breaking characteristics. Without wishing to be bound by any particular theory, it is believed that the emulsion inhibiting and breaking characteristics of the binary polyethylenimine composition may be caused by the combination of diffusion power and charge density. The combination of two different molecular weight polyethylenimine—a comparatively low molecular weight polyethylenimine and a comparatively high molecularly weight polyethylenimine—each of which are in the low to midsize molecular weight range of possible polyethylenimine structures allows good diffusion into liquids being treated. Further, the hyperbranched structure of each polyethylenimine forming the binary polyethylenimine composition (e.g., which may be unmodified and non-alkoxylated) leads to a high charge density. High charge density means that the molecule carries a significant electrical charge allowing it to effectively interact with and destabilize the interface between oil droplets and water and promote separation.

The first hyperbranched polyethylenimine having a comparatively low molecular weight may exhibit a cationic charge density greater than 5 meq/g, such as greater than 10 meq/g, greater than 12.5 meq/g, greater than 15 meq/g, greater than 17.5 meq/g, or greater than 20.0 meq/g. For example, the first hyperbranched polyethylenimine may exhibit a cationic charge density within a range from 10 meq/g to 30 meq/g, such as from 15 meq/g to 25 meq/g, or from 18 meq/g to 22 meq/g.

The second hyperbranched polyethylenimine having a comparatively high molecular weight may exhibit a cationic charge density greater than 2 meq/g, such as greater than 3 meq/g, greater than 4 meq/g, greater than 5 meq/g, or greater than 6 meq/g. For example, the second hyperbranched polyethylenimine may exhibit a cationic charge density within a range from 1 meq/g to 10 meq/g, such as from 3 meq/g to 8 meq/g, from 4 meq/g to 7 meq/g.

The ratio of the weight of the first hyperbranched polyethylenimine having a comparatively low molecular to a combined weight of the first and second hyperbranched polyethylenimine components in the binary polyethylenimine composition may range from 0.1 to 0.8, such as from 0.125 to 0.5, from 0.15 to 0.4, from 0.2 to 0.35, or from 0.25 to 0.3. Correspondingly, the ratio of the weight of the second hyperbranched polyethylenimine having a comparatively high molecular to a combined weight of the first and second hyperbranched polyethylenimine components in the binary polyethylenimine composition may range 0.9 to 0.2, such as from 0.875 to 0.5, from 0.85 to 0.6, from 0.8 to 0.65, or from 0.75 to 0.7.

The binary polyethylenimine composition may include additional constituent components in addition to the active first and second hyperbranched polyethylenimine components. When configured with additional components, the first and second hyperbranched polyethylenimine components may constitute at least 2 wt % of the binary polyethylenimine composition, such as at least 5 wt % of the binary polyethylenimine composition, or at least 10 wt % of the binary polyethylenimine composition. For example, the first and second hyperbranched polyethylenimine components may range from 2 wt % to 20 wt % of the binary polyethylenimine composition, such as from 3 wt % to 10 wt %. In some examples, the first hyperbranched polyethylenimine having a comparatively low molecular weight is within a range from approximately 1 wt % to approximately 3 wt % of the binary polyethylenimine composition and the second hyperbranched polyethylenimine having a comparatively high molecular is within a range from approximately 2.5 wt % to approximately 7.5 wt % of the binary polyethylenimine composition.

When the binary polyethylenimine composition includes additional constituent components, a remainder of the weight of the binary polyethylenimine composition may be or include water. For example, water may be at least 50 wt % of the composition, such as at least 60 wt %, at least 70 wt %, at least 80 wt %, at least 85 wt %, or at least 90 wt %. Other example constitutes that may be in the composition include antipolymerants (also referred to as polymerization inhibitors), hydrocarbon solvent (e.g., pytar solvent), corrosion inhibitors dispersants, and the like and combinations thereof.

The binary polyethylenimine composition can effectively inhibit the formation of, and/or resolve (break) an emulsion across a range of dosing concentrations, including low dosing concentrations. The actual amount of the binary polyethylenimine composition used may depend, for example, on the amount of the first and second hyperbranched polyethylenimine components in the binary polyethylenimine composition compared to other constituent components. The binary polyethylenimine composition may be dosed in an amount effective to achieve a concentration of the combined first and second hyperbranched polyethylenimine components within a range from 0.01 ppm to 30 ppm, preferably from 0.1 to 10 ppm, more preferably from 0.5 to 2 ppm, and all value or ranges there between (e.g., 0.02, 0.03, 0.04, 0.05, 0.06, 0.07, 0.08, 0.09, 0.1, 0.15, 0.2, 0.3, 0.4, 0.5, 1, 2, 3, 4, 5, 6, 7, 8, or 9 ppm). That is, the binary polyethylenimine composition may be dosed so the combined amount of the first and second hyperbranched polyethylenimine components is within any one of the foregoing ranges or at any one of the foregoing amounts, with the relative amount of the first and second hyperbranched polyethylenimine components depending on the relative ratios of the different components in the binary polyethylenimine composition, as discussed above.

In practice, the pH of the hydrocarbon-containing water being treated (e.g., quench water) with the binary polyethylenimine composition may vary (e.g., over time at a specific location and/or between different locations). The binary polyethylenimine composition can effectively inhibit the formation of, and/or resolve (break) an emulsion across a range of pH conditions. The pH of the liquid being treated that the binary polyethylenimine composition is effective at treating may be within a pH range from 2 to 12, such as from 4 to 10, 5 to 9, or 6 to 8. While the temperature of the hydrocarbon-containing water being treated may vary, the temperature may typically be within a range from 25 to 150° C., such as from 50 to 90° C.

The binary polyethylenimine composition can be contacted and/or mixed with a quench water to prevent the formation of an emulsion and/or to resolve the quench water composition into two immiscible phases. The quench water may be used in the production of alkenes (e.g., ethylene, propylene, butylene, etc.). While example quench water systems are described, it should be understood that the binary polyethylenimine composition can be used in other hydrocarbon/water systems, such as hydrocarbon/water systems from hydrocarbon production from a subsurface formation, and the disclosure is not otherwise limited in this respect.

The quench water composition can be any aqueous petrochemical composition applicable to quenching. The quench water composition may include an organic phase and an aqueous phase that are immiscible with each other, where the binary polyethylenimine composition promotes the longevity of this immiscibility during continued recycling and reuse of the water. The binary polyethylenimine composition can be added to a feed stream entering a quench water component, directly to the component, or both. The organic phase can include gasoline hydrocarbons and the aqueous phase can be transferred to a process water stripper (PWS), a dilution steam generator preheater and/or a dilution steam generator.

The quench water treated with the binary polyethylenimine composition can include hydrocarbons such as ethylene, propylene, butylene, butadiene, benzene, toluene, xylene, and combinations thereof. The binary polyethylenimine composition can be added to the quench water, resulting in formation of an organic phase and an aqueous phase that are immiscible with each other. The after addition of the binary polyethylenimine composition to separate the organic phase and the aqueous phase from each other, the immiscible phase and the aqueous phase can be separated from each other (e.g., using a decanter or other gravity settling separator), and the separated aqueous phase recycled for reuse as quench water.

The binary polyethylenimine composition can be used to decrease the turbidity of the aqueous phase of the quench water composition as compared to prior to treatment with the binary polyethylenimine composition. The residual turbidity level of the aqueous phase of the quench water may be reduced by at least 25% after treatment with 1 ppm of the first and second hyperbranched polyethylenimine components (that is, an amount of the binary polyethylenimine composition may be effective to achieve a concentration of the combined first and second hyperbranched polyethylenimine components of 1 ppm), such as at least 35%, at least 45%, at least 50%, at least 55%, at least 65%, at least 75%, at least 85%, or at least 95%. The residual turbidity level of the aqueous phase of the quench water after such treatment may be less than 1500 Nephelometric Turbidity Units (NTU), such as less than 1200 NTU, less than 1000 NTU, less than 800 NTU, less than 600 NTU, less than 400 NTU, less than 300 NTU, less than 200 NTU, or less than 100 NTU.

The following examples may provide additional details about binary polyethylenimine compositions according to the disclosure.

### Example 1: Evaluation of Emulsion Breaking Performance Across a Range of Dosage and pH Conditions

A binary polyethylenimine composition according to the disclosure was formulated from first and second hyperbranched polyethylenimine components selected as follows:

| First hyperbranched | Second hyperbranched |
|---|---|
| polyethylenimine | polyethylenimine |
| component | component |
| Lupasol ® G-20, | EPOMIN ™ P-3000, |
| commercially available | commercially available |
| from BASF, | from Nippon Shokubai, |
| CAS # 25987-06-8 | CAS # 9002-98-6 |
| | (Aziridine homopolymer) |
| MW 1,300 g/mol | MW 450,000 g/mol |

The binary polyethylenimine composition was formulated with a concentration of approximately 3 wt % of the first hyperbranched polyethylenimine component and approximately 7.5 wt % of the second hyperbranched polyethylenimine component, based on the total weight of the binary polyethylenimine composition.

The emulsion breaking performance of the binary polyethylenimine composition was evaluated by measuring turbidity reduction in a test water sample. Turbidity measurements were carried out using the HACH Turbidity meter (2100Q/TL 2300), using 10 ml of test sample. Measurements were carried out before and after adding the emulsion breaker.

For comparison purposes, the emulsion breaking performance of other alternative treatment options were evaluated using the same test protocol and conditions used to measure the emulsion breaking performance of the binary polyethylenimine composition. Alternative treatment options studied where (1) Lupasol® G-20 alone, (2) EPOMIN™ P-3000 alone, and (3) a “Comparison Product” formulated from a high molecular weight (approx. 2M g/mol) modified PEI and a medium molecular weight (approx. 28K to 110K g/mol) ethoxylated PEI.

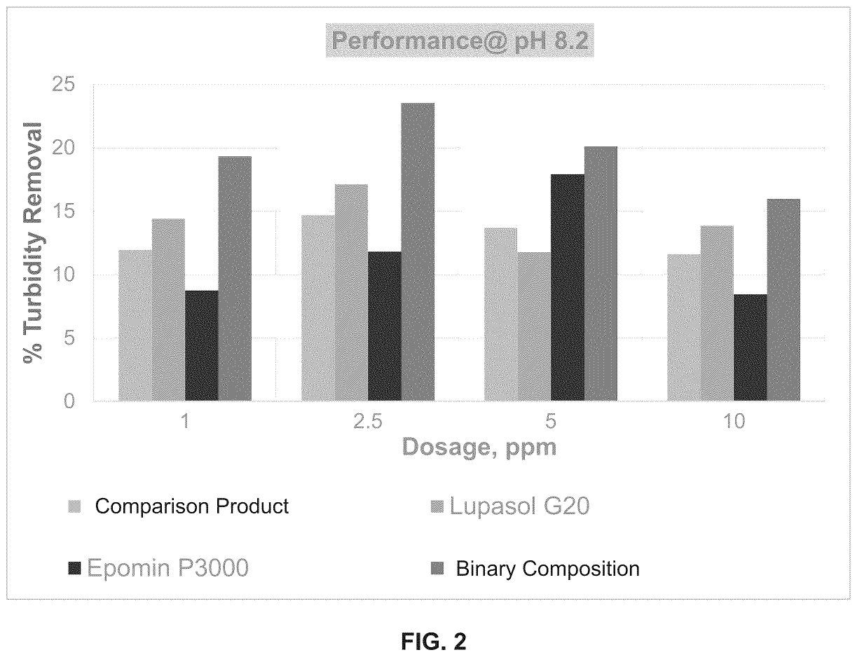

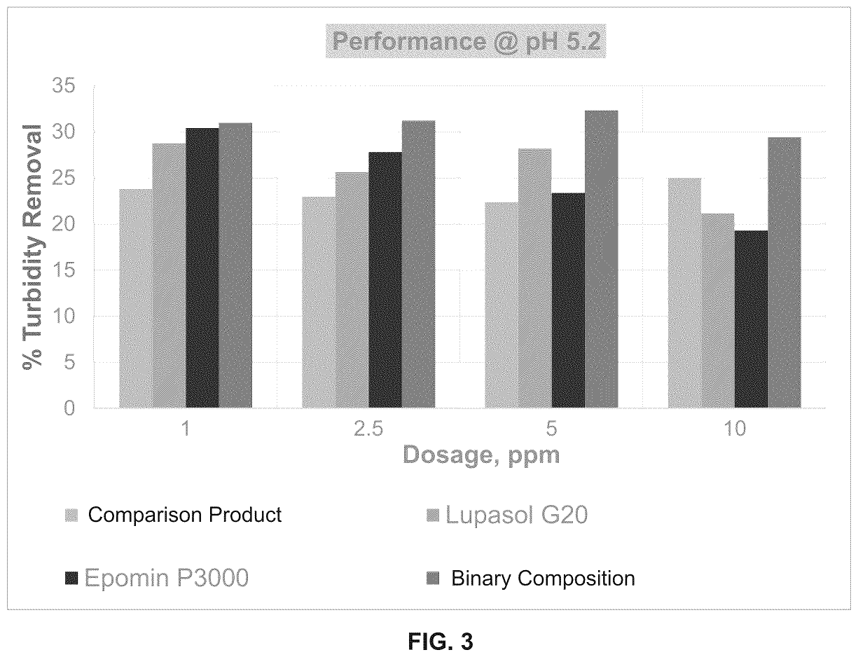

FIGS. 2 and 3 are bar charts showing the turbidity removal performance of the example binary polyethylenimine composition compared to alternative emulsion treatment chemistry options across a range of dosage concentrations. FIG. 2 illustrates performance results at a test water pH of 8.2. FIG. 3 illustrates performance results at a test water pH of 5.2. The data illustrate the superior performance of the binary polyethylenimine composition across a range of pH conditions that may be observed in commercial practice as well as across a range of commercial dosage levels relative to the individual constituent hyperbranched polyethylenimine components forming the composition as well as competing treatment alternatives. The data illustrate synergistic performance of the binary polyethylenimine composition exceeding the performance of the individual constituent hyperbranched polyethylenimine components forming the composition.

Dosages points in FIGS. 2 and 3 are expressed in parts per million (ppm) unit as the product/formulation. The alternative emulsion treatment options evaluated had at least 3× higher weight percent PEI components than the binary polyethylenimine composition.

### Example 2: Evaluation of Physical Properties and Different Emulsion Breaking Chemicals

The physical properties—specifically charge density, molecular weight, amine type distribution—of different emulsion breaker compositions were evaluated to assess property trends relative to emulsion breaking performance. Tables 1A (at an acidic pH) and 1B (at a basic pH) below report cationic charge density for the different components studied. Table 2 below reports molecular weight for the different components studied. Table 3 below reports amine type distribution for the different components studied.

TABLE 1A

| | | Charge | Soln. | | |
|---|---|---|---|---|---|
| | | Density | Conc. | Native pH | |
| SN | Component | (meq/g) | (ppm) | Native | Adjusted |
| 1 | High Molecular Weight | 8.43 | 100 | 7.78 | 4.26 |
| | (approx. 2M g/mol) | | | | |
| | modified PEI | | | | |
| 2 | Medium Molecular Weight | 0.27 | 100 | 7.92 | 4.53 |
| | (approx. 28K to 110K | | | | |
| | g/mol) ethoxylated PEI | | | | |
| 3 | Lupasol ® G20 | 18.74 | 100 | 9.83 | 4.52 |
| | | 20.16 | 50 | | 4.63 |
| | | 22.69 | 25 | | 4.55 |
| 4 | Epomin ™ P3000 | 6.77 | 100 | 8.17 | 4.49 |
| 5 | Comparison Product | 1.28 | 100 | 7.92 | 4.55 |
| | Discussed in Example 1 | | | | |
| 6 | Binary PEI Composition | 10.51 | 100 | 8.08 | 4.56 |
| | According to the | | | | |
| | Disclosure (Lupasol ® G20 + | | | | |
| | Epomin ™ P3000) | | | | |

TABLE 1B

| | | Charge | Soln. | | |
|---|---|---|---|---|---|
| | | Density | Conc. | pH | |
| SN | Component | (meq/g) | (ppm) | Native | Adjusted |
| 1 | High Molecular Weight | 7.84 | 100 | 7.78 | No |
| | (approx. 2M g/mol) | | | | |
| | modified PEI | | | | |
| 2 | Medium Molecular Weight | 0.42 | 100 | 7.92 | No |
| | (approx. 28K to 110K | | | | |
| | g/mol) ethoxylated PEI | | | | |
| | Lupasol ® G20 | 17.78 | 100 | 9.83 | 8.08 |
| 4 | Epomin ™ P3000 | 4.45 | 100 | 8.17 | 8.05 |
| 5 | Comparison Product | 1.18 | 100 | 7.92 | No |
| | Discussed in Example 1 | | | | |
| 6 | Binary PEI Composition | 8.02 | 100 | 8.08 | 8 |
| | According to the | | | | |
| | Disclosure (Lupasol ® G20 + | | | | |
| | Epomin ™ P3000) | | | | |

TABLE 2

| | High | Medium | | |
|---|---|---|---|---|
| | Molecular | Molecular | | |
| | Weight | Weight | | |
| | modified | ethoxylated | | Epomin |
| Component | PEI | PEI | Lupasol G20 | P3000 |
| MW (g/mole) | 2,000,000 | 28000 to | 1300 | 450,000 |
| | | 110,000 | | |

TABLE 3

| | | | Medium | High |
|---|---|---|---|---|
| | | | Molecular | Molecular |
| | | | Weight | Weight |
| | Lupasol ® | Epomin ™ | ethoxylated | modified |
| Sample/Amine Type | G20 | P3000 | PEI | PEI |
| Primary amine | 37% | 32% | 0% | 46% |
| Secondary amine | 37% | 41% | 17% | 30% |
| Tertiary amine | 26% | 27% | 83% | 24% |

The data indicated a trend for increasing emulsion breaking performance with increasing cationic charge density. The data also indicate a trend of increased diffusion power with decreasing molecular weight.

Various examples have been described. These and other examples are within the scope of the following claims.

1. A method of treating ethylene plant quench water, the method comprising:
adding a binary polyethylenimine composition to a quench water for an ethylene production process thereby inhibiting and/or breaking an emulsion in the quench water, wherein the binary polyethylenimine composition comprises a first hyperbranched polyethylenimine having a first molecular weight and a second hyperbranched polyethylenimine having a second molecular weight greater than the first molecular weight.

2. The method of claim 1, wherein the first molecular weight is less than 50,000 g/mol and the second molecular weight is greater than 100,000 g/mol.

3. The method of claim 1, wherein first molecular weight is different from the second molecular weight by at least one order of magnitude.

4. The method of claim 1, wherein adding the binary polyethylenimine composition to the quench water comprises adding the binary polyethylenimine composition to the quench water within a pH range from 5 to 9.

5. The method of claim 1, wherein the first hyperbranched polyethylenimine and the second hyperbranched polyethylenimine are each unmodified.

6. The method of claim 1, wherein:
the first hyperbranched polyethylenimine and the second hyperbranched polyethylenimine each comprise a primary amine fraction, a secondary amine fraction, and a tertiary amine fraction; and
the primary amine fraction, the secondary amine fraction, and the tertiary amine fraction are each present in a molar percentage within a range from 20% to 50% of all amine groups present in each of the first hyperbranched polyethylenimine and the second hyperbranched polyethylenimine, respectively.

7. The method of claim 6, wherein:
the primary amine fraction is present in a molar percentage within a range from 30% to 40% of all amine groups present in each of the first hyperbranched polyethylenimine and the second hyperbranched polyethylenimine, respectively;
the second amine fraction is present in a molar percentage within a range from 35% to 50% of all amine groups present in each of the first hyperbranched polyethylenimine and the second hyperbranched polyethylenimine, respectively; and
the tertiary amine fraction is present in a molar percentage within a range from 20% to 30% of all amine groups present in each of the first hyperbranched polyethylenimine and the second hyperbranched polyethylenimine, respectively.

8. The method of claim 1, wherein the first hyperbranched polyethylenimine exhibits a charge density within a range from 15 to 25 meq/g, and the second hyperbranched polyethylenimine exhibits a charge density within a range from 4 to 7 meq/g.

9. The method of claim 1, wherein the first molecular weight is within a range from 1000 to 50,000 g/mol and the second molecular weight is within a range from 100,000 g/mol to 1,000,000 g/mol.

10. The method of claim 1, wherein adding the binary polyethylenimine composition to the quench water comprises adding an amount of the binary polyethylenimine composition to the quench water effective to achieve a concentration of a combination of the first hyperbranched polyethylenimine and the second hyperbranched polyethylenimine within a range from 0.1 ppm to 10 ppm.

11. The method of claim 1, further comprises separating the quench water into an aqueous phase and an immiscible phase after addition of the binary polyethylenimine composition, and separating the immiscible phase and the aqueous phase from each other.

12. The method of claim 1, wherein a residual turbidity level of the aqueous phase is less 400 NTU after addition of 1 ppm of the binary polyethylenimine composition to the quench water.

13. The method of claim 1, wherein adding the binary polyethylenimine composition to the quench water comprises adding the binary polyethylenimine composition to a feed of one or more of a quench water tower, a quench water loop, and/or a quench water settler.

14. The method of claim 1, wherein the quench water comprises benzene, styrene, and butadiene.

15. An emulsion treatment composition for ethylene plant quench water, the emulsion treatment composition comprising:
a first hyperbranched polyethylenimine having a first molecular weight; and
a second hyperbranched polyethylenimine having a second molecular weight greater than the first molecular weight.

16. The composition of claim 15, wherein the first molecular weight is less than 50,000 g/mol and the second molecular weight is greater than 100,000 g/mol.

17. The composition of claim 15, wherein first molecular weight is different from the second molecular weight by at least one order of magnitude.

18. The composition of claim 15, wherein the first hyperbranched polyethylenimine and the second hyperbranched polyethylenimine are each unmodified.

19. The composition of claim 15, wherein:
the first hyperbranched polyethylenimine and the second hyperbranched polyethylenimine each comprise a primary amine fraction, a secondary amine fraction, and a tertiary amine fraction; and
the primary amine fraction, the secondary amine fraction, and the tertiary amine fraction are each present in a molar percentage within a range from 20% to 50% of all amine groups present in each of the first hyperbranched polyethylenimine and the second hyperbranched polyethylenimine, respectively.

20. The composition of claim 19, wherein:
the primary amine fraction is present in a molar percentage within a range from 30% to 40% of all amine groups present in each of the first hyperbranched polyethylenimine and the second hyperbranched polyethylenimine, respectively;
the second amine fraction is present in a molar percentage within a range from 35% to 50% of all amine groups present in each of the first hyperbranched polyethylenimine and the second hyperbranched polyethylenimine, respectively; and
the tertiary amine fraction is present in a molar percentage within a range from 20% to 30% of all amine groups present in each of the first hyperbranched polyethylenimine and the second hyperbranched polyethylenimine, respectively.

21. The composition of claim 15, wherein the first hyperbranched polyethylenimine exhibits a charge density within a range from 15 to 25 meq/g, and the second hyperbranched polyethylenimine exhibits a charge density within a range from 4 to 7 meq/g.

22. The composition of claim 15, wherein the first molecular weight is within a range from 1000 to 50,000 g/mol and the second molecular weight is within a range from 100,000 g/mol to 1,000,000 g/mol.
